(19) United States
(12) Patent Application Publication
(10) Pub. No.: US 2004/0241877 A1
(43) Pub. Date: Dec. 2, 2004

(54) Test strips for the detection of prion proteins

(76) Inventors: Paul Price, Zurich (CH); Bruno Oesch, Stilli (CH); Eric Kubler, Basel (CH)

Correspondence Address: RANKIN, HILL, PORTER & CLARK LLP, 4080 ERIE STREET, WILLOUGHBY, OH, 44094-7836, US

(21) Appl. No.: 10/490,003

(22) PCT Filed: Sep. 24, 2002
(86) PCT No.: PCT/EP02/10681

(30) Foreign Application Priority Data
Sep. 25, 2001 (DE) 101 47 012.6

(51) Int. Cl. G01N 33/558

(52) U.S. Cl. 436/514

(57) ABSTRACT

A test strip for detecting an analyte in liquid or homogenised samples, with a section which may be brought into contact with the sample and with at least one first defined region on the test strip in which detection reagents are immobilised which bind the prion protein. A device is also provided that permits simultaneous testing of several samples in sample containers, provided in a group with a defined geometrical arrangement. The device includes a holder in which several test strips are fixed in an arrangement corresponding to the defined geometrical arrangement of the sample containers, such that the lower section thereof may be introduced into each of the sample containers.

### BACKGROUND OF THE INVENTION

The invention relates to a test strip for detecting an analyte in a liquid and a holder allowing several test strips to be introduced into different sample containers.

Test strips are being used for the detection of a multitude of different analytes in liquid or homogenized samples. In general, the test strips are provided with at least one defined region, in which a detection reagent for a certain analyte is immobilized.

Usually, a lower section of the test strips is contacted with the sample. The defined detection region resides either in the lower section of the test strip and is then directly wetted by the sample. According to another option, the detection region is provided farther up on the test strip, though this requires that the strip is made from a liquid-conducting material and the sample liquid can flow from the contact section to the detection region under the effect of capillary action.

Whether or not a test is positive or negative may be observed e.g. by means of a color change in the detection region.

Usually, test strips are used especially in detection applications that are particularly well-suited for standardization and/or which can be performed at home by otherwise untrained individuals, etc. Known examples include e.g. pregnancy tests or test strips for the monitoring of renal function.

In contrast, the detection or prion proteins requires extensive preparations, the performance of which is restricted at this time to highly specialized personnel working in safety laboratories. The detection reaction itself, which is usually performed separately for each sample, is associated with relatively little effort as compared to the sample preparation, which is the reason why standardization and simplification have not been seriously considered at this time.

On the other hand, considering the growing number of tests, e.g. on slaughtered animals, it cannot be excluded that standardization of the reaction for the detection of prion proteins may well afford substantial savings in terms of both labor and costs.

Thus, it is the task of the present invention to provide a device allowing for substantially simpler detection of prions in liquid, liquefied or homogenized samples than possible at this time. It is another task of the present invention to provide a device allowing for particularly simple simultaneous measurement of several samples.

### SUMMARY OF THE INVENTION

The tasks, as stated, are solved by a test strip with at least a first defined region in which detection reagents specifically binding to proteins are immobilized, and by a device having a holder in which several test strips are held in an arrangement that corresponds to the defined geometrical arrangement of sample containers such that a lower section of each test strip can be introduced simultaneously into one of the sample containers.

Similar to known test strips for other analytes, the test strip according to the invention comprises a lower section which can be contacted with a liquid or homogenized sample, whereby, according to the invention, a first defined region is provided, in which antibodies or other detection reagents binding to the prion protein are immobilized. Suitable antibodies are known to the expert in this field from the publication by Korth, C. et al., Nature 1997, vol. 390, pages 74-77.

In accordance with the invention, the test strip is made from absorbent material, in particular from nitrocellulose or polyvinylidene fluoride (PVDF), so that the transport of liquid between the lower section, which can be contacted with the sample, and the first defined region is possible.

In a common test with the test strip according to the invention, a detection reagent, e.g. antibodies, is added to the sample initially in order to form, in the presence of a marker, a detectable complex with the prion protein. Markers such as radioactive isotopes, fluorescent substances, chromophores absorbing UV or visible light can be used for detection. In the latter case, such markers may comprise e.g. dyed polymer beads, such as latex beads, gold particles, liposomes, and dye particles or similar substances. As a matter of principle, all detectable markers which bind or can bind actively or passively to antibodies or other detection reagents are suitable for this purpose. For simplicity, only examples of antibodies bound to dyed markers shall be described in the following.

Subsequently, the test strip is contacted with the sample and the sample liquid migrates across the first defined region, in which the detection reagents immobilized therein bind to the prion protein and colored marker attached thereto contained in the sample. This leads to a color change in the first defined region, which may be detected, e.g. by eye.

Suitable detection reagents include e.g. antibodies, aptamers or other means specifically recognizing prion proteins.

In order to check whether or not the detection reaction proceeded properly, the test strip may be provided with one or several other defined regions, in which control reagents are immobilized.

In this context, the test strip according to a preferred embodiment comprises a second defined region, in which reagents capable of binding the colored reagent-marker complex in the sample are immobilized. This allows a check to be made on whether the reagent-marker complex was truly added to the sample and/or whether the concentrations are correct, etc. The control reagents used for this purpose may comprise especially an antibody or a functional reagent, which bind to the colored reagent-marker complex only, if the complex is explicitly capable of binding to the prion protein. In particular, recombinant prior protein may be used as the control reagent.

Another preferred embodiment concerns test strips for use in PrP^{Sc} detection reactions. Prion proteins are known to exist in two different isoforms denoted PrP^{c} and PrP^{Sc}. PrP^{c} is the isoform of a normal mammalian protein, whereas PrP^{Sc} is an anomalous, pathological isoform.

Currently, it is being presumed that PrP^{Sc} is specific for prion diseases. Common tests and detection procedures are based on the presumption that PrP^{Sc} is a marker of disease and thus test for the presence of this molecule in samples.

However, this procedure is associated with the problem that samples from infected sources usually do not contain PrP^{Sc} exclusively, but also PrP^{c}. Thus, the detection procedure must differentiate between the commonly present PrP^{c} and the possibly present PrP^{SC}.

Currently, this differentiation is afforded by digesting the sample with a protease making use of the fact that the PrP^{c} form is completely digestible, whereas only an N-terminal section of the PrP^{Sc} form is protease-sensitive, while a section denoted PrP 27-30 is not digested.

Thus, in a specific PrP^{Sc} assay, antibodies specifically binding to PrP 27-30 are immobilized in the first defined region of a test strip according to the invention, although it cannot be excluded that a corresponding region of the PrP^{c} form would be bound upon incomplete digestion.

In order to be sure in this regard, another preferred embodiment may provide the test strip according to the invention with a third defined region, in which control reagents are immobilized which specifically recognize and bind to the N-terminus of the PrP protein. If the digestion proceeds to completion, the N-terminal region of PrP should be no longer detectable. In contrast, a color change in this region indicates the presence of residual intact PrP^{c} in the sample which means that the digestion was incomplete. Suitable control reagents include e.g. the antibodies known from the publication of Barry, R. A. et al.; J. Immunol. 1988, vol. 140, pages 1188-1193.

This arrangement provides for particularly simple means for checking whether or not the digestion was complete simultaneous to the detection of prions, whereas state-of-the-art ELISA procedures required a separate test for this purpose.

Moreover, the test strip according to the invention may be provided with a region denoted as “waste pad” to take up the liquid that flowed through the strip. This region may be provided e.g. with an absorbent mat or a fleece or blotting paper or similar means. The shape and depth of the test strip may be designed in such a way that a small chromatography column is formed which allows for the separation and detection of PrP even in large volumes.

The test strips with waste pads according to the invention as described above are preferably made from absorbent material to allow the liquid to flow from the sample to the, possibly separate, detection and control regions.

Obviously, it is also conceivable to use test strips not made of absorbent material. In this case, the defined detection and, possible, control regions would have to be provided on the test strip in such a way that they could be directly wetted with the sample or the liquid would have to be moved actively (e.g. by means of suction/aspiration or centrifugation).

Aside from the tests strips mentioned above, the invention comprises a device for the simultaneous testing of multiple samples.

Usually, multiple samples are simultaneously processed by use of microtiter plates or by means of other formats combining several sample containers into a composite system with a defined geometrical arrangement.

Thus, the preferred device according to the invention comprises a holder, in which several test strips are arranged and taken up in an oriented fashion in such a way that their lower sections can be introduced simultaneously into the sample containers, with one test strip being introduced per sample container.

Therefore, a strip-shaped holder is conceivable, in which the test strips are fixed parallel to each other and at a certain distance from each other with the distance corresponding to the distance of the sample containers in the row of a microtiter plate.

Another embodiment of the invention provides a holder which takes up all test strips required for a certain format simultaneously. For instance, a holder for a microtiter plate is conceivable, in which as many test strips are taken up as corresponds to the number of sample container wells.

In a particularly preferred embodiment, a device of this kind might comprise e.g. a frame, in which one stripe-shaped holder with a corresponding number of test strips can be introduced for each row of the microtiter plate. To read the results, the holders can be removed successively from the frame, which facilitates the analysis.

### BRIEF DESCRIPTION OF THE DRAWINGS

In the following, the invention is illustrated in detail by means of several figures depicting the different embodiments. Additional figures show the results of tests, in which the test strips according to the invention were used. As such,

FIG. 1 shows a test strip;

FIG. 2 shows an embodiment of the holder, in which several test strips are attached parallel to each other to form a row;

FIG. 3 shows a frame in the common microtiter plate format, in which the holders shown in FIG. 2 can be placed;

FIG. 4 shows a frame completely fitted with holders according to FIG. 2;

FIG. 5 shows the results of a test, in which several test strips attached in a holder such as the one shown in FIG. 2 were used for the detection of recombinant bovine prion protein at various concentrations;

FIG. 6 shows the results of a test with several test strips for the detection of cellular prion protein in transgenic mice and wild types;

FIG. 7 shows the results of a test with two test strips for the detection of disease-specific, protease-resistant prion protein, and

FIG. 8 shows the results of a test with three test strips for checking the digestion conditions of BSE homogenate.

### DETAILED DESCRIPTION OF THE PREFERRED EMBODIMENTS

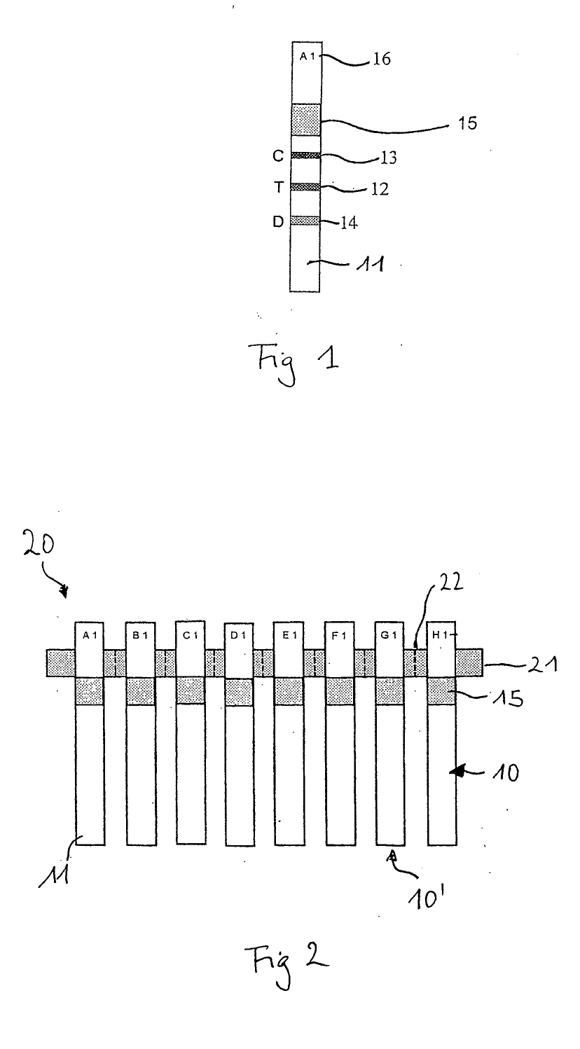

FIG. 1 shows a test strip, 10, for the detection of prion proteins. The test strip comprises a lower section, 11, which can be contacted with a homogenized or liquid sample.

Moreover, several defined regions, 12, 13, and 14 are provided on test strip 10, each of which contains detection or control reagents. These reagents may be applied to test strip 10 for instance by spraying.

Test strop 10 consists of absorbent material, e.g. nitrocellulose. Sample liquid contacting test strip 10 in section 11 is aspirated through the test strip along regions 14, 12, 13 to waste pad 15, which takes up the liquid after its flow through the test strip. An identification, 16, is provided at the upper end of the test strip to indicate e.g. the coordinates of the sample in a microtiter plate.

As mentioned above, different reagents are fixed in the defined regions, 12, 13, and 14. It is mandatory for any test strip to contain reagents which recognize any prion protein that may be present in the sample. In the case shown, these reagents are specific antibodies against the prion protein and the reagents are contained in defined region 12.

Region 13 contains control reagents allowing the concentration and/or presence of the colored detection reagent-marker complex mentioned above to be checked in the sample.

And lastly, region 14 contains reagents allowing the digestion of prion proteins to be checked.

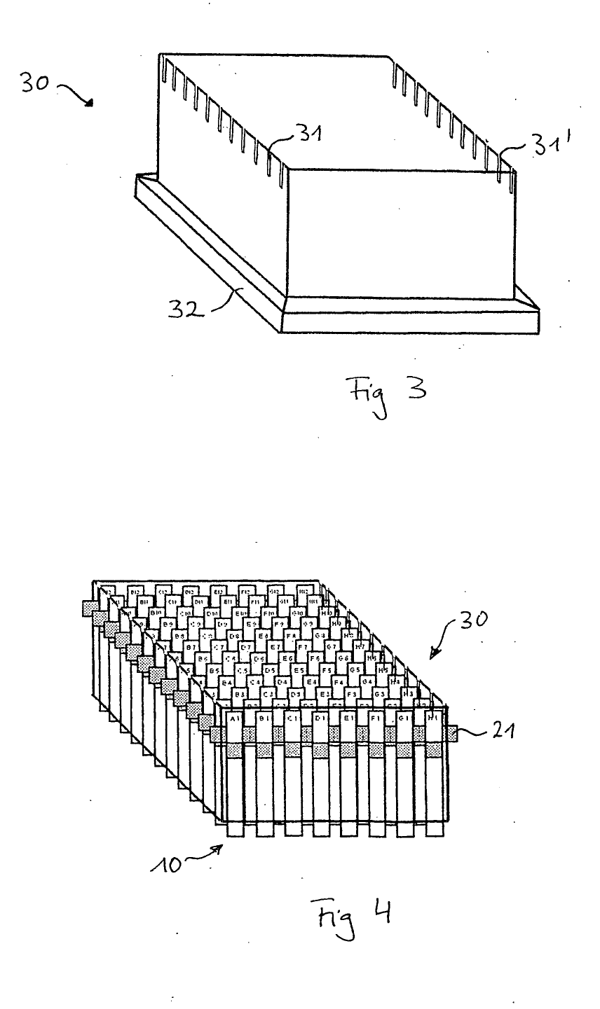

FIG. 2 shows a device, 20, in which multiple samples can be analyzed simultaneously. Device 20 comprises a holder, 21, in which test strips 10, 10′, etc., are taken up in a parallel arrangement with their lower sections, 11, pointing downwards. The mutual distance between test strips 10, 10′ is selected in such a way that it corresponds to the usual distance of wells in a microtiter plate. Using holder 21, samples in the wells of a row of a microtiter plate can be checked simultaneously.

Perforations 22 facilitating the separation of individual test strips 10, 10′ may be provided between the individual test strips, 10, 10′ on holder 21 which takes the shape of a strip in the case shown.

It is conceivable to extend this format to the entire microtiter plate.

In this context, FIG. 3 shows frame 30, whose base is selected in such a way that the frame can be placed on a conventional microtiter plate by means of an adapter, 32, so that the plate is completely covered by the frame. In the area of the upper edges, 30, which extend in a longitudinal direction, mutually opposite pairs of slits, 31, 31′, are provided, into which one holder, 21, each with test strips 10, 10′ can be placed.

The number of opposite pairs of slits 31, 31′, corresponds to the number of rows in a microtiter plate so that one holder 21 each can be introduced per row of the microtiter plate.

Holder 30 in its fully assembled state is shown in FIG. 4, in which adapter 32 is not shown.

The shown test strips each comprise a defined region, 12, 13, 14, containing the different detection and control reagents. It is self-evident that the invention also considers embodiments, in which several, rather than one, defined regions are provided on the test strip for each reagent, i.e. two or more defined regions each bearing detection reagents capable of detecting prion protein present in the sample, or two or more regions containing reagents for checking the digestion, etc.

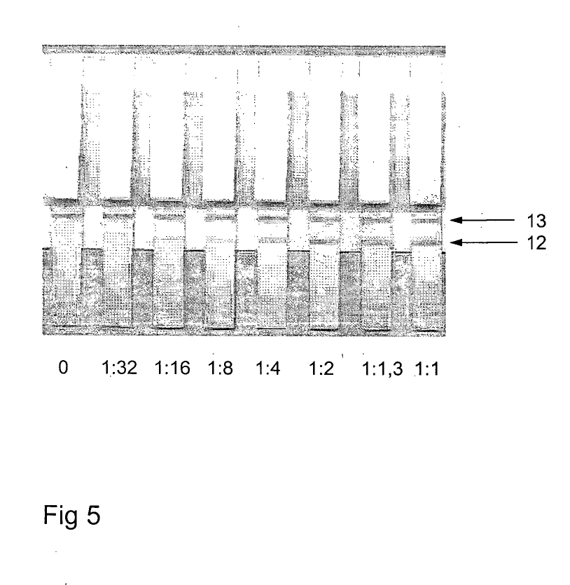

FIG. 5 shows the results of a test with several test strips for the detection of recombinant bovine prion protein (RecBoPrP) at various concentrations. The test strips were incubated with various starting concentrations of RecBoPrP in accordance with the methods presented above. The sample dilution was in the range from 1:1 to 1:32. The respective dilution is indicated in the figure below the test strip. As a blank control, a test strip was incubated with a sample containing no RecBoPrP.

Whereas region 13 of the test strips responding to the presence of the detection reagentmarker complex in the sample is constantly colored, region 12 of the test strips recognizing PrP 27-30 shows decreasing color intensity corresponding to the decreasing concentration of RecBoPrP. The difference in the color intensity of region 12 of the test strips over the concentration range (32-fold) is easily detectable by eye. Therefore, the test strips can also be used for semi-quantitative detection of RecBoPrP.

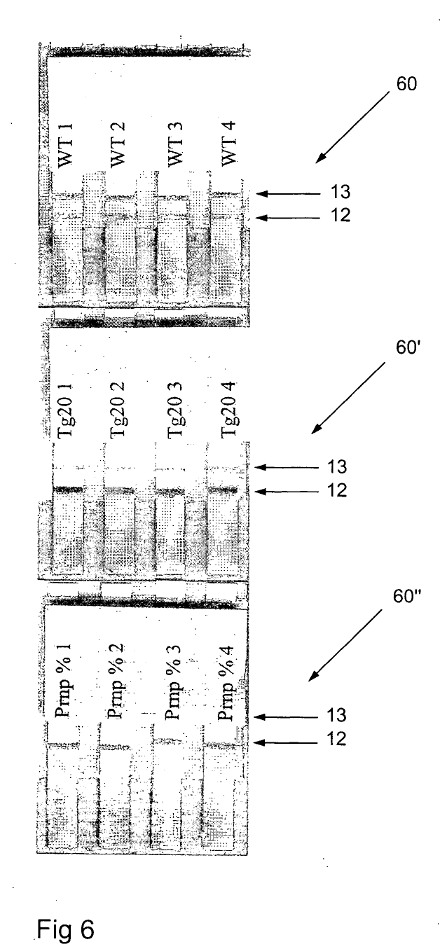

FIG. 6 shows three rows, 60, 60′, and 60″, with several test strips each after incubation with urine from four transgenic or wildtype mice.

As before, the test strips comprise region 12 recognizing PrP 27-30 and region 13 responding to the presence of the detection reagent-marker complex in the sample.

The test strips of row 60 were incubated with urine from four wildtype mice (WT1-WT4) producing normal quantities of prion protein which resulted in substantial coloring of region 12 of the test strips.

The test strips of row 60′ were incubated with urine from four transgenic mice (Tg20 1-Tg20 4) producing strongly elevated quantities of prion protein. This leads to region 12 of the test strips being even more strongly colored.

The test strips of row 60″ were incubated with urine from four transgenic mice (Prnp % 1-Prnp % 4) producing no prion protein. Consequently, region 12 of the test strips is not colored.

An abbreviation above each test strip in the figure allows the identification of the mouse to which the test strip corresponds.

It is evident from this figure that the test strips described above specifically recognize the prion protein even when it is present in a complex environment (urine).

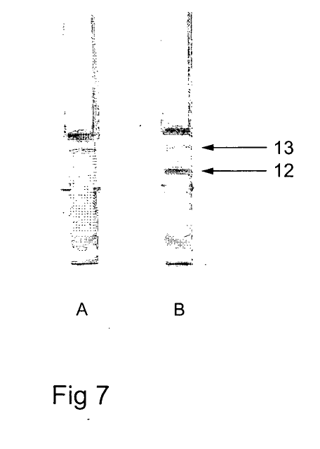

FIG. 7 shows test strips A and B used to analyze protease-treated brain homogenate from a healthy cow versus a cow afflicted by BSE.

As mentioned above, the non-infectious isofonn of the prion protein (PrP^{c}) is completely digested by protease treatment, whereas the infectious isoform (PrP^{Sc}) is only partially digested so that a domain denoted PrP 27-30 remains.

As before, test strips A and B comprise region 12 recognizing PrP 27-30 and region 13 responding to the presence of the detection reagent-marker complex in the sample.

Test strip A was incubated with protease-treated brain homogenate of a healthy cow. Region 12 of the test strip remains non-colored, since PrP^{c} was digested to completion.

Test strip B was incubated with protease-treated brain homogenate of a cow afflicted by BSE. It is evident that region 12 of the test strip is colored. This means that PrP 27-30 is present despite protease treatment indicating that the sample contained PrP^{Sc} prior to the digestion.

Therefore, a strip test of the type shown herein is well-suited for rapid BSE screening of bovine brain samples.

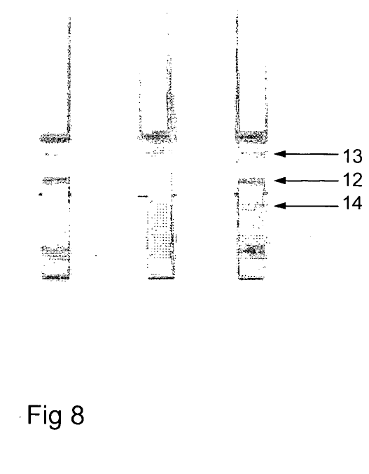

FIG. 8 shows test strips A, B, and C, after incubation with different homogenates which had been digested to different degrees.

Aside from regions 12 recognizing PrP 27-30 and regions 13 responding to the presence of the detection reagent-marker complex in the sample, test strips A, B, and C comprise regions 14 which can bind to the N-terminal region of the prion protein and thus recognize undigested or incompletely digested prion protein exclusively, but not digested prion protein lacking its N-terminal region.

Test strip A was incubated with completely digested, protease-treated brain homogenate of a cow afflicted by BSE. Region 12 is colored since the homogenate contains PrP 27-30, whereas region 14 remains non-colored, because the complete digestion leads to the absence of N-terminal regions.

Test strip B was incubated with completely digested, protease-treated brain homogenate of a healthy cow. This sample contains neither PrP 27-30 nor N-terminal regions resulting in regions 12 and 14 remaining non-colored.

Test strip C was incubated with incompletely digested, protease-treated brain homogenate of a healthy cow. Region 12 of this strip is colored, because the poor digestion causes the homogenate to still contain the PrP 27-30 domain, which is also present in PrP^{°}. Region 14 is colored because there are still some N-terminal regions present. If there were no region 14 in the test strip, it would not have been possible in this case to safely differentiate whether the coloration of region 12 may be an indication of the presence of PrP 27-30 in a positive sample or instead be related to incomplete digestion of normal prion protein.

1. A test strip for the detection of an analyte in liquid, liquefied or homogenized samples, said test strip comprising a section (11) that may be brought into contact with the sample and at least a first defined region (12), whereby the test strip (10) consist of an absorbent material, and wherein detection reagents specifically binding to the prion protein are immobilized in the first defined region (12).

2. The test strip according to claim 1, wherein the test strip consists of nitrocellulose or PVDF.

3. The test strip according to claim 1, wherein said test strip includes a second defined region (13) in which a control reagent binding to a colored detection reagent-marker complex present in the sample is immobilized.

4. The test strip according to claim 3, wherein antibodies binding to the detection reagents in the colored detection reagent-marker complex are bound in the second defined region (13).

5. The test strip according to claim 3, wherein said test strip includes a third defined region (14) in which a control reagent is immobilized, said third defined region being used to check whether prion protein digestion has been successful.

6. The test strip according to claim 5, wherein a detection reagent recognizing an N-terminus of the prion protein is immobilized in the third defined region (14).

7. The test strip according to claim 5, wherein the first defined region (12) is arranged between the second (13) and the third defined regions (14).

8. The test strip according to claim 5, wherein the defined regions are provided in a section of the test strip that is directly wetted by sample liquid when the test strip contacts the sample.

9. A device simultaneously testing multiple samples in sample containers that are combined into a composite system with a defined geometrical arrangement, wherein the device comprises a holder (21), in which several test strips (10, 10′) are held in an arrangement that corresponds to the defined geometrical arrangement of the sample containers such that a lower section (11) of each test strip can be introduced simultaneously into one of the sample containers each.

10. The device according to claim 7, wherein the holder (21) is designed in the form of a transverse strip on which the test strips are arranged in an orthogonal direction.

11. The device according to claim 10, further comprising a frame (30) that can be placed on a conventional microtiter plate, said frame providing opposite receptacles (31, 31′) in the area of its opposite edges, in which the strip-shaped holder (21) can be introduced.
